(19) United States
(12) Patent Application Publication
(10) Pub. No.: US 2026/0007301 A1
(43) Pub. Date: Jan. 8, 2026

(54) MEDICAL IMAGE PROCESSING DEVICE, MEDICAL IMAGE PROCESSING METHOD, AND COMPUTER-READABLE RECORDING MEDIUM

(71) Applicant: OLYMPUS MEDICAL SYSTEMS CORP., Tokyo (JP)

(72) Inventor: Masahide YAMAKI, Tokyo (JP)

(73) Assignee: OLYMPUS MEDICAL SYSTEMS CORP., Tokyo (JP)

(21) Appl. No.: 19/327,103

(22) Filed: Sep. 12, 2025

Related U.S. Application Data
(63) Continuation of application No. PCT/JP2023/027444, filed on Jul. 26, 2023.
(60) Provisional application No. 63/454,752, filed on Mar. 27, 2023.

(51) Int. Cl. A61B 1/00; G06T 5/70; G16H 40/63

(52) U.S. Cl. CPC A61B 1/00009; A61B 1/00006; A61B 1/00059; G06T 5/70; G16H 40/63; G06T 2207/10068; G06T 2207/30004

(57) ABSTRACT

A medical image processing device includes a processing module performing data processing with respect to imaging data, the processing module being capable of implementing a first algorithm which enables data processing with respect to first imaging data obtained as a result of imaging performed by a first endoscope, and a second algorithm which enables data processing with respect to the first imaging data and with respect to second imaging data obtained as a result of imaging performed by a second endoscope, the processing module being configured to determine type of endoscope that is connected, when connection with the first endoscope is determined, perform data processing according to an algorithm that is selected as a result of a user operation from among the first algorithm and the second algorithm, and when connection with the second endoscope is determined, perform data processing according to the second algorithm.

### CROSS REFERENCES TO RELATED APPLICATIONS

This application is a continuation of International Application No. PCT/JP2023/027444, filed on Jul. 26, 2023 which claims the benefit of priority of U.S. Provisional Application No. 63/454,752 filed on Mar. 27, 2023, the entire contents of which are incorporated herein by reference.

### BACKGROUND

### 1. Technical Field

The present disclosure relates to a medical image processing device, a medical image processing method, and a computer-readable recording medium.

### 2. Related Art

In the related art, an endoscope system is known that includes: an endoscope which is equipped with an imaging device that is inserted inside a body and that performs imaging of the inside of the body; and a medical image processing device to which the endoscope is connected in a detachably-attachable manner and which is equipped with a processing module for processing the imaging data obtained as a result of imaging performed by the endoscope.

There are times when a variety of endoscopes of different product generations are connectible to a single medical image processing device. The variety of endoscopes differ in: the type of imaging device (a charge coupled device (CCD) or a complementary metal oxide device (CMOS)); the pixel count of the imaging device; the sensitivity of the imaging device; and the features of the optical system.

Regarding a medical image processing device that is compatible to a variety of endoscopes of different product generations, it is possible to think of a first configuration and a second configuration given below.

In the first configuration, the algorithm to be implemented for data processing is changed according to each endoscope that gets connected; and data processing of the imaging data, which is obtained as a result of imaging performed by the connected endoscope, is performed according to the corresponding algorithm and display images (endoscope images) are generated.

In the second configuration, regardless of the endoscope that is connected from among a variety of endoscopes; data processing of the imaging data, which is obtained as a result of imaging performed by the connected endoscope, is performed according to a common algorithm and endoscope images are generated (for example, refer to Japanese Patent Application H8-238216).

### SUMMARY

In some embodiments, a medical image processing device includes a processing module performing data processing with respect to imaging data which is input, the processing module being capable of implementing a first algorithm which enables data processing with respect to first imaging data obtained as a result of imaging performed by a first endoscope, and a second algorithm which enables data processing with respect to the first imaging data and with respect to second imaging data obtained as a result of imaging performed by a second endoscope, the processing module being configured to determine type of endoscope that is connected, when connection with the first endoscope is determined, perform data processing according to an algorithm that is selected as a result of a user operation from among the first algorithm and the second algorithm, and when connection with the second endoscope is determined, perform data processing according to the second algorithm.

In some embodiments, a medical image processing device includes: a processing module performing data processing with respect to imaging data obtained as a result of imaging performed by a medical observation device; and an operation receiver receiving a user operation, processing module being configured to, with respect to first image data obtained as a result of imaging performed by a first medical observation device from among medical observation devices, perform data processing according to an algorithm that is selected in the user operation from among a first algorithm dedicated for the first medical observation device, and a second algorithm that is an algorithm of a latter design generation than the first algorithm and that is compatible to the first medical observation device and to a second medical observation device which is the medical observation device of a latter product generation than the first medical observation device, and with respect to second image data obtained as a result of imaging performed by the second medical observation device, perform data processing according to the second algorithm.

In some embodiments, provided is a medical image processing method implemented in a medical image processing device. The method includes: with respect to first imaging data which is obtained as a result of imaging performed by a first medical observation device, performing data processing according to an algorithm selected in a user operation from among a first algorithm dedicated for the first medical observation device, and a second algorithm that is an algorithm of a latter design generation than the first algorithm and that is compatible to the first medical observation device and to a second medical observation device which is a latter product generation than the first medical observation device; and with respect to second imaging data which is obtained as a result of imaging performed by the second medical observation device, performing data processing according to the second algorithm.

In some embodiments, provided is a non-transitory computer-readable recording medium with an executable program stored thereon. The program causes a medical image processing device to execute: with respect to first imaging data which is obtained as a result of imaging performed by a first medical observation device, performing data processing according to an algorithm selected in a user operation from among a first algorithm dedicated for the first medical observation device, and a second algorithm that is an algorithm of a latter design generation than the first algorithm and that is compatible to the first medical observation device and to a second medical observation device which is a latter product generation than the first medical observation device; and with respect to second imaging data which is obtained as a result of imaging performed by the second medical observation device, performing data processing according to the second algorithm.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating a configuration of an endoscope system according to an embodiment;

FIG. 2 is a block diagram illustrating a configuration of a control device;

FIG. 3 is a diagram for explaining a first modification example of the embodiment;

FIG. 4 is a diagram for explaining a second modification example of the embodiment;

FIG. 5 is a diagram for explaining a third modification example of the embodiment;

FIG. 6 is a diagram for explaining a fourth modification example of the embodiment;

FIG. 7 is a diagram for explaining a fifth modification example of the embodiment; and

FIG. 8 is a diagram for explaining a sixth modification example of the embodiment.

### DETAILED DESCRIPTION

An illustrative embodiment (hereinafter, called an embodiment) according to the embodiment is described below with reference to the accompanying drawings. However, the disclosure is not limited by the embodiment described below. Moreover, in the drawings, identical constituent elements are referred to by the same reference numerals.

### Overall Configuration of Endoscope System

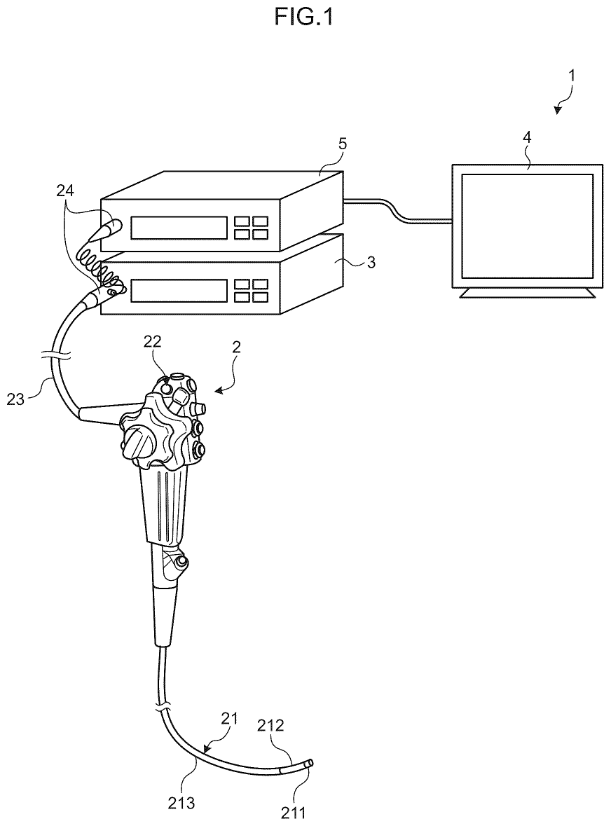

FIG. 1 is a diagram illustrating a configuration of an endoscope system 1 according to the embodiment.

The endoscope system 1 is used in the medical field and represents a system for observing the inside of a biological object. As illustrated in FIG. 1, the endoscope system 1 includes an endoscope 2, a light source device 3, a display 4, and a control device 5.

The endoscope 2 corresponds to a medical observation device.

In the present embodiment, the endoscope 2 is, what is called, a flexible endoscope. When partially inserted inside a biological object, the endoscope 2 performs imaging of inside of the biological object and outputs an image signal obtained as a result of performing the imaging. As illustrated in FIG. 1, the endoscope 2 includes an insertion portion 21, an operating unit 22, a universal cord 23, and a connector unit 24.

The insertion portion 21 is at least partially flexible and is inserted inside a biological object. As illustrated in FIG. 1, the insertion portion 21 includes a front end unit 211, a curved portion 212, and a flexible tube 213.

The front end unit 211 is disposed at the front end of the insertion portion 21. Although not specifically illustrated in FIG. 1, an illumination optical system, an imaging optical system, and an imaging unit are installed in the front end unit 211.

The illumination optical system faces one end of a light guide (not illustrated) that is drawn inside the insertion portion 21; and emits an observation light, which is transmitted by the light guide, into the biological object from the front end of the insertion portion 21.

The imaging optical system takes in the feedback light (a subject image) of the observation light that was emitted into the biological object from the illumination optical system, and forms an image on the imaging surface of an imaging device constituting the imaging unit.

The imaging unit is configured using an imaging device such as a CCD or a CMOS; and performs imaging of the subject image formed by the imaging optical system and outputs an imaging signal generated as a result of performing the imaging. In the following explanation, the imaging signal is referred to as imaging data.

The curved portion 212 is linked to the proximal end of the front end unit 211 (i.e., the side toward the operating unit 22). Although not specifically illustrated in FIG. 1, the curved portion 212 is configured by linking a plurality of curved pieces, and hence becomes bendable.

The flexible tube 213 is linked to the proximal end of the curved portion 212 (i.e., the side toward the operating unit 22), and is formed as a flexible and long tube.

The operating unit 22 is connected to the proximal end portion of the insertion portion 21. The operating unit 22 receives various operations that are performed with respect to the endoscope 2.

The universal cord 23 extends from the operating unit 22 in a different direction than the direction of extension of the insertion portion 21; and has the light guide arranged thereon and has a signal line arranged thereon for transmitting the operation signals.

The connector unit 24 is disposed at the end portion of the universal cord 23, and is connected to the light source device 3 and the control device 5 in a detachably-attachable manner.

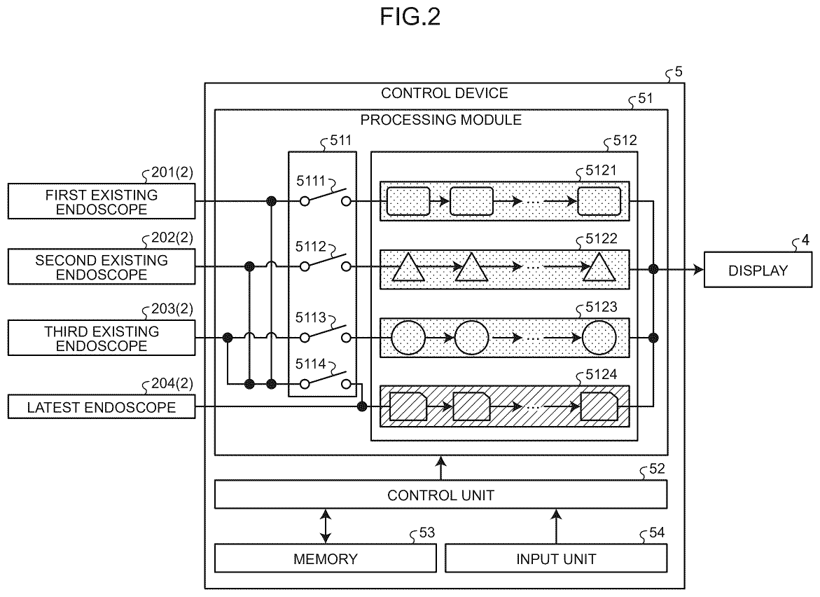

In the present embodiment, a variety of endoscopes 2 of different product generations are configured to be connectible to the light source device 3 and the control device 5. In the following explanation, of the variety of endoscopes 2, existing endoscopes 2 are referred to as a first existing endoscope 201 to a third existing endoscope 203 (see FIG. 2). Moreover, of the variety of endoscopes 2, the latest type of endoscope 2 is referred to as a latest endoscope 204 (see FIG. 2).

The latest endoscope 204 either is an endoscope of the same product generation as the control device 5 or is an endoscope released around the same time when the software representing the essential part of the control device 5 was updated. The latest endoscope 204 corresponds to a second medical observation device. In the following explanation, the imaging data obtained as a result of imaging performed by the latest endoscope 204 is referred to as latest imaging data. That latest imaging data corresponds to second imaging data.

The third existing endoscope 203 is an endoscope released one product generation earlier (for example, three to five years earlier) than the product generation of the latest endoscope 204. In other words, the third existing endoscope 203 either is an endoscope of one product generation earlier than the product generation of the control device 5 or is an endoscope released one generation earlier than the time when the software representing the essential part of the control device 5 was updated. The third existing endoscope 203 corresponds to a first medical observation device. In the following explanation, the imaging data obtained as a result of imaging performed by the third existing endoscope 203 is referred to as third existing imaging data. The third existing imaging data corresponds to first imaging data.

The second existing endoscope 202 is an endoscope released one product generation earlier than the product generation of the third existing endoscope 203. In other words, the second existing endoscope 202 either is an endoscope of two product generations earlier than the product generation of the control device 5 or is an endoscope released two generations earlier than the time when the software representing the essential part of the control device 5 was updated. The second existing endoscope 202 corresponds to the first medical observation device. In the following explanation, the imaging data obtained as a result of imaging performed by the second existing endoscope 202 is referred to as second existing imaging data. The second existing imaging data corresponds to the first imaging data.

The first existing endoscope 201 is an endoscope released one product generation earlier than the product generation of the second existing endoscope 202. In other words, the first existing endoscope 201 either is an endoscope of three product generations earlier than the product generation of the control device 5 or is an endoscope released three generations earlier than the time when the software representing the essential part of the control device 5 was updated. The first existing endoscope 201 corresponds to the first medical observation device. In the following explanation, the imaging data obtained as a result of imaging performed by the first existing endoscope 201 is referred to as first existing imaging data. The first existing imaging data corresponds to the first imaging data.

The first existing endoscope 201, the second existing endoscope 202, the third existing endoscope 203, and the latest endoscope 204 differ in: the type of imaging device (CCD or CMOS); the pixel count of the imaging device; the sensitivity of the imaging device; and the features of the optical system.

Under the control performed by the control device 5, the light source device 3 supplies the observation light to the other end of the light guide. The observation light passes through the light guide and the illumination optical system, and gets emitted toward the inside of the biological object from the front end of the insertion portion 21. Examples of the observation light include the white light (the normal light) representing a broadband light; an excitation light representing a narrow-band light meant for causing excitation of a fluorescent agent such as indocyanine green; and a narrow-band light used in narrow band imaging (NBI).

The display 4 is a liquid crystal display (LCD) or an organic electroluminescence (EL) display in which, under the control performed by the control device 5, a display image (an endoscope image) is displayed that corresponds to a video signal output from the control device 5.

The control device 5 corresponds to a medical image processing device. The control device 5 includes a controller such as a central processing unit (CPU) or a micro processing unit (MPU), and comprehensively controls the operations of the endoscope 2 (the imaging unit) and the light source device 3. However, the control device 5 is not limited to include a CPU or an MPU, and can alternatively include an application specific integrated circuit (ASIC), a field-programmable gate array (FPGA), or a graphics processing unit (GPU).

Regarding the detailed configuration of the control device 5, the explanation is given below in the section “Configuration of control device”.

### Configuration of Control Device

Given below is the explanation about a configuration of the control device 5.

FIG. 2 is a block diagram illustrating a configuration of the control device 5.

As illustrated in FIG. 2, the control device 5 includes a processing module 51, a control unit 52, a memory 53, and an input unit 54.

The processing module 51 performs data processing with respect to the imaging data (RAW data) obtained as a result of imaging performed by the endoscope 2, and generates a video signal corresponding to a display image to be displayed in the display 4. As illustrated in FIG. 2, the processing module 51 includes a switching portion 511 and a data processing portion 512.

Under the control performed by the control unit 52, the switching portion 511 switches the output destination for the imaging data (RAW data) that is obtained from the endoscope 2. As illustrated in FIG. 2, the switching portion 511 includes a first for-existing switching portion 5111, a second for-existing switching portion 5112, a third for-existing switching portion 5113, and a common switching portion 5114.

The first for-existing switching portion 5111 switches between outputting and not outputting the first existing imaging data (RAW data), which is obtained as a result of imaging performed by the first existing endoscope 201, to a first for-existing data processing portion 5121 (see FIG. 2) of the data processing portion 512. In the following explanation, for explanatory convenience, as the states of the first for-existing switching portion 5111, the state in which the first existing imaging data (RAW data) is output to the first for-existing data processing portion 5121 is treated as the “ON” state; and the state in which no data is output to the first for-existing data processing portion 5121 is treated as the “OFF” state.

The second for-existing switching portion 5112 switches between outputting and not outputting the second existing imaging data (RAW data), which is obtained as a result of imaging performed by the second existing endoscope 202, to a second for-existing data processing portion 5122 (see FIG. 2) of the data processing portion 512. In the following explanation, for explanatory convenience, as the states of the second for-existing switching portion 5112, the state in which the second existing imaging data (RAW data) is output to the second for-existing data processing portion 5122 is treated as the “ON” state; and the state in which no data is output to the second for-existing data processing portion 5122 is treated as the “OFF” state.

The third for-existing switching portion 5113 switches between outputting and not outputting the second existing imaging data (RAW data), which is obtained as a result of imaging performed by the third existing endoscope 203, to a third for-existing data processing portion 5123 (see FIG. 2) of the data processing portion 512. In the following explanation, for explanatory convenience, as the states of the third for-existing switching portion 5113, the state in which the third existing imaging data (RAW data) is output to the third for-existing data processing portion 5123 is treated as the “ON” state; and the state in which no data is output to the third for-existing data processing portion 5123 is treated as the “OFF” state.

The common switching portion 5114 switches between outputting and not outputting the first existing imaging data (RAW data), or the second existing imaging data (RAW data), or the third existing imaging data (RAW data) to a common data processing portion 5124 (see FIG. 2) of the data processing portion 512. In the following explanation, for explanatory convenience, as the states of the common switching portion 5114, the state in which the first existing imaging data (RAW data), or the second existing imaging data (RAW data), or the third existing imaging data (RAW data) is output to the common data processing portion 5124 is treated as the “ON” state; and the state in which no data is output to the common data processing portion 5124 is treated as the “OFF” state.

The data processing portion 512 performs data processing with respect to the imaging data (RAW data) input thereto. The data processing portion 512 is configured using an FPGA or a GPU. As illustrated in FIG. 2, the data processing portion 512 includes the first for-existing data processing portion 5121, the second for-existing data processing portion 5122, the third for-existing data processing portion 5123, and the common data processing portion 5124.

The first for-existing data processing portion 5121 performs data processing with respect to the first existing imaging data (RAW data), which is input thereto, according to a dedicated algorithm corresponding to the first existing endoscope 201, and generates a video signal based on the first existing imaging data. That algorithm corresponds to a first algorithm.

The data processing performed by the first for-existing data processing portion 5121 includes resolution adjustment, contrast adjustment, noise reduction, and brightness correction (gain correction) corresponding to the first existing endoscope 201. Moreover, the data processing includes image processing according to the display characteristics of the display 4 and includes, for example, color adjustment according to a user operation of the input unit 54. Examples of the display characteristics of the display 4 include the color balance and the gamma curve of the display 4. Meanwhile, the data processing can also include optical black reduction (clamping), white balance adjustment, color reconstruction, color correction matrix processing, gamma correction, and YC processing for converting RGB signals into luminance and chrominance signals (Y, Cb/Cr signals).

The second for-existing data processing portion 5122 performs data processing with respect to the second existing imaging data (RAW data), which is input thereto, according to a dedicated algorithm corresponding to the second existing endoscope 202, and generates a video signal based on the second existing imaging data. That algorithm corresponds to the first algorithm.

The data processing performed by the second for-existing data processing portion 5122 includes resolution adjustment, contrast adjustment, noise reduction, and brightness correction (gain correction) corresponding to the second existing endoscope 202. Moreover, the data processing includes image processing according to the display characteristics of the display 4 and includes, for example, color adjustment according to a user operation of the input unit 54. Examples of the display characteristics of the display 4 include the color balance and the gamma curve of the display 4. Meanwhile, the data processing can also include optical black reduction (clamping), white balance adjustment, color reconstruction, color correction matrix processing, gamma correction, and YC processing for converting RGB signals into luminance and chrominance signals (Y, Cb/Cr signals).

The third for-existing data processing portion 5123 performs data processing with respect to the second existing imaging data (RAW data), which is input thereto, according to a dedicated algorithm corresponding to the third existing endoscope 203, and generates a video signal based on the third existing imaging data. That algorithm corresponds to the first algorithm.

The data processing performed by the third for-existing data processing portion 5123 includes resolution adjustment, contrast adjustment, noise reduction, and brightness correction (gain correction) corresponding to the third existing endoscope 203. Moreover, the data processing includes image processing according to the display characteristics of the display 4 and includes, for example, color adjustment according to a user operation of the input unit 54. Examples of the display characteristics of the display 4 include the color balance and the gamma curve of the display 4. Meanwhile, the data processing can also include optical black reduction (clamping), white balance adjustment, color reconstruction, color correction matrix processing, gamma correction, and YC processing for converting RGB signals into luminance and chrominance signals (Y, Cb/Cr signals).

The common data processing portion 5124 performs data processing with respect to the input imaging data, from among the first existing imaging data to the third existing imaging data (RAW data) and the latest imaging data (RAW data), according to an algorithm that is of a latter design generation than the first algorithm mentioned above and that is compatible to all endoscopes 2 including the first existing endoscope 201 to the third existing endoscope 203 and the latest endoscope 204; and generates a video signal based on the imaging data. Herein, the algorithm represents the latest algorithm designed around the same time when the control device 5 was released or when the software representing the essential part of the control device 5 was updated; and corresponds to a second algorithm.

The data processing performed by the common data processing portion 5124 includes resolution adjustment, contrast adjustment, noise reduction, and brightness correction (gain correction) corresponding mainly to the latest endoscope 204. As explained above, the data processing is performed according to the algorithm that is compatible to all endoscopes 2 including the first existing endoscope 201 to the third existing endoscope 203 and the latest endoscope 204. For that reason, resolution adjustment, contrast adjustment, noise reduction, and brightness correction (gain correction) is applicable also to the first existing imaging data, the second existing imaging data, and the third existing imaging data. Moreover, the data processing includes image processing according to the display characteristics of the display 4 and includes, for example, color adjustment according to a user operation of the input unit 54. Examples of the display characteristics of the display 4 include the color balance and the gamma curve of the display 4. Meanwhile, the data processing can also include optical black reduction (clamping), white balance adjustment, color reconstruction, color correction matrix processing, gamma correction, and YC processing for converting RGB signals into luminance and chrominance signals (Y, Cb/Cr signals).

In FIG. 2, for explanatory convenience, the constituent elements that perform data processing according the algorithm that was used in the past (according to the first algorithm) (i.e., the first for-existing data processing portion 5121 to the third for-existing data processing portion 5123) are illustrated using dots; and the constituent element that performs data processing according to the algorithm applicable to all endoscopes 2 including the first existing endoscope 201 to the third existing endoscope 203 and the latest endoscope 204 (according to the second algorithm) (i.e., the common data processing portion 5124) is illustrated using oblique lines.

The control unit 52 is implemented when a controller such as a CPU or an MPU executes various computer programs stored in the memory 53; and controls the operations performed by the endoscope 2 (the imaging unit) and the light source device 3 as well as controls the operations of the entire control device 5. However, the control unit 52 is not limited to be configured using a CPU or an MPU, and can alternatively be configured using an ASIC or an FPGA.

Regarding the functions of the control unit 52, the explanation is given later in “Operations performed by control device”.

The memory 53 stores therein the computer programs (including a medical image processing program), which are executed by the control unit 52, and the information required in the operations performed by the control unit 52.

The input unit 54 corresponds to an operation receiver. The input unit 54 is configured using an operation device such as a mouse, a keyboard, or a touch-sensitive panel; and receives a user operation. Then, the input unit 54 outputs an operation signal, which corresponds to the user operation, to the control unit 52.

### Operations Performed by Control Device

Given below is the explanation of the operations performed by (i.e., a medical image processing method implemented by) the control device 5.

In the following explanation, the operations performed when the user uses the first existing endoscope 201, the operations performed when the user uses the second existing endoscope 202, the operations performed when the user uses the third existing endoscope 203, and the operations performed when the user uses the latest endoscope 204 are explained in that order.

Operations Performed when User Uses First Existing Endoscope

Firstly, the explanation is given about the operations performed when the user uses the first existing endoscope 201. Thus, in this case, from among the first existing endoscope 201, the second existing endoscope 202, the third existing endoscope 203, and the latest endoscope 204; only the first existing endoscope 201 is connected to the control device 5.

At the time of performing data processing (implementing a data processing step) with respect to the first existing imaging data (RAW data) obtained as a result of imaging performed by the first existing endoscope 201, the processing module 51 performs the following operations.

As a result of a user operation of the input unit 54, when the first algorithm is selected from among the first algorithm and the second algorithm, under the control performed by the control unit 52, the first for-existing switching portion 5111 switches to the “ON” state and the common switching portion 5114 switches to the “OFF” state. As a result, the first existing imaging data (RAW data) output from the first existing endoscope 201 is input to the first for-existing data processing portion 5121. Then, the first for-existing data processing portion 5121 implements the first algorithm to perform data processing with respect to the first existing imaging data (RAW data), and generates a video signal. Subsequently, a display image based on the video signal is displayed in the display 4.

On the other hand, as a result of a user operation of the input unit 54, when the second algorithm is selected from among the first algorithm and the second algorithm, under the control performed by the control unit 52, the first for-existing switching portion 5111 switches to the “OFF” state and the common switching portion 5114 switches to the “ON” state. As a result, the first existing imaging data (RAW data) output from the first existing endoscope 201 is input to the common data processing portion 5124. Then, the common data processing portion 5124 implements the second algorithm to perform data processing with respect to the first existing imaging data (RAW data), and generates a video signal. Subsequently, a display image based on the video signal is displayed in the display 4.

Operations Performed when User Uses Second Existing Endoscope

Given below is the explanation about the operations performed when the user uses the second existing endoscope 202. Thus, in this case, from among the first existing endoscope 201, the second existing endoscope 202, the third existing endoscope 203, and the latest endoscope 204; only the second existing endoscope 202 is connected to the control device 5.

At the time of performing data processing (implementing a data processing step) with respect to the second existing imaging data (RAW data) obtained as a result of imaging performed by the second existing endoscope 202, the processing module 51 performs the following operations.

Regarding the operations performed by the processing module 51 when the user uses the second existing endoscope 202, with reference to the explanation given above about “the operations performed when the user uses the first existing endoscope 201”; “the first for-existing switching portion 5111” can be substituted with “the second for-existing switching portion 5112”, “the first existing endoscope 201” can be substituted with “the second existing endoscope 202”, “the first existing imaging data (RAW data)” can be substituted with “the second existing imaging data (RAW data)”, and “the first for-existing data processing portion 5121” can be substituted with “the second for-existing data processing portion 5122”.

Operations Performed when User Uses Third Existing Endoscope

Given below is the explanation about the operations performed when the user uses the third existing endoscope 203. Thus, in this case, from among the first existing endoscope 201, the second existing endoscope 202, the third existing endoscope 203, and the latest endoscope 204; only the third existing endoscope 203 is connected to the control device 5.

At the time of performing data processing (implementing a data processing step) with respect to the third existing imaging data (RAW data) obtained as a result of imaging performed by the third existing endoscope 203, the processing module 51 performs the following operations.

Regarding the operations performed by the processing module 51 when the user uses the third existing endoscope 203, with reference to the explanation given above about “the operations performed when the user uses the first existing endoscope 201”; “the first for-existing switching portion 5111” can be substituted with “the third for-existing switching portion 5113”, “the first existing endoscope 201” can be substituted with “the third existing endoscope 203”, “the first existing imaging data (RAW data)” can be substituted with “the third existing imaging data (RAW data)”, and “the first for-existing data processing portion 5121” can be substituted with “the third for-existing data processing portion 5123”.

Operations Performed when User Uses Latest Endoscope

Given below is the explanation about the operations performed when the user uses the latest endoscope 204. Thus, in this case, from among the first existing endoscope 201, the second existing endoscope 202, the third existing endoscope 203, and the latest endoscope 204; only the latest endoscope 204 is connected to the control device 5.

At the time of performing data processing (implementing a data processing step) with respect to the latest imaging data (RAW data) obtained as a result of imaging performed by the latest endoscope 204, the processing module 51 performs the following operations.

The latest imaging data (RAW data) that is output from the latest endoscope 204 is directly input to the common data processing portion 512 without involving the switching portion 511. The common data processing portion 5124 implements the second algorithm to perform data processing with respect to the latest imaging data (RAW data), and generates a video signal. Then, a display image based on the video signal is displayed in the display 4.

According to the present embodiment described above, the following effects are achieved.

The control device 5 according to the present embodiment implements the algorithm that is selected according to the user operation from among the first algorithm dedicated to the first existing endoscope 201 and the second algorithm compatible to the first existing endoscope 201 and the latest endoscope 204; and performs data processing with respect to the first existing imaging data that is obtained as a result of imaging performed by the first existing endoscope 201. The same is the case regarding the second existing imaging data and the third existing imaging data obtained as a result of imaging performed by the second existing endoscope 202 and the third existing endoscope 203, respectively. On the other hand, with respect to the latest imaging data obtained as a result of imaging performed by the latest endoscope 204, the control device 5 performs data processing according to the second algorithm.

Thus, it becomes possible to generate endoscope images that are preferred by a first user as well as a second user, and to enhance the user-friendliness. A first user is a user who, even when the control device 5 is updated, if the endoscope 2 of an old generation is used, prefers generation of endoscope images having the exact same appearance as the appearance achieved in the case of using a pre-update control device. A second user is a user who, when the control device 5 is updated, even if the endoscope 2 of an old generation is used, prefers generation of endoscope images that are subjected to data processing by the updated control device 5 using the latest algorithm.

### OTHER EMBODIMENTS

Till now, the embodiment was described. However, the disclosure is not limited to the embodiment described above.

Alternatively, it is possible to implement a first modification example to a sixth modification example explained below.

### First Modification Example

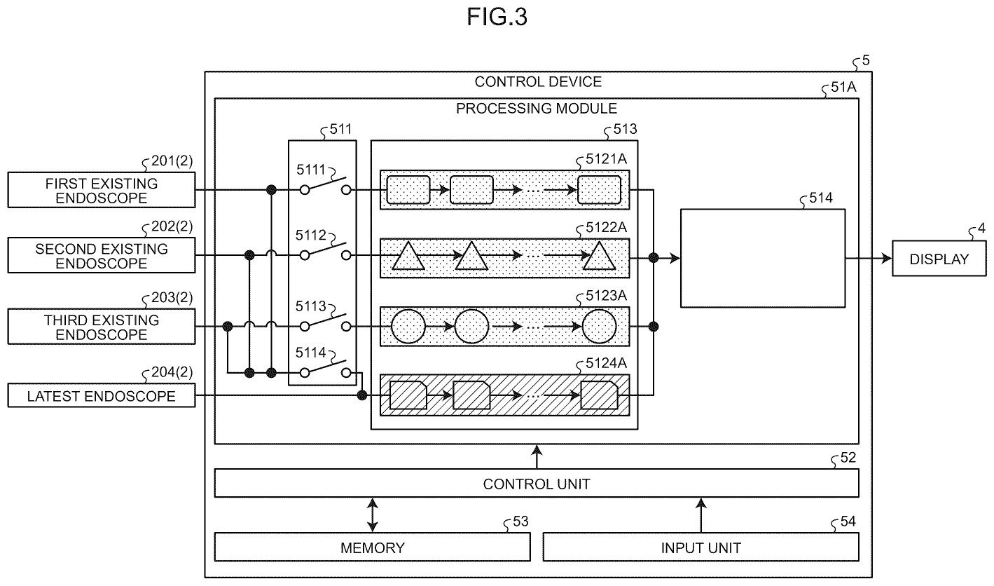

FIG. 3 is a diagram for explaining the first modification example of the embodiment. More particularly, FIG. 3 is a block diagram corresponding to FIG. 2.

In the embodiment described above, the configuration of the processing module 51 can be changed to the configuration according to the first modification example as illustrated in FIG. 3. In the following explanation, for explanatory convenience, the processing module 51 according to the first modification example is referred to as a processing module 51A.

As illustrated in FIG. 3, the processing module 51A includes a first data processing portion 513 and a second data processing portion 514 in addition to including the switching portion 511 according to the embodiment described above. The first data processing portion 513 and the second data processing portion 514 are configured using an FPGA or a GPU. For example, the first data processing portion 513 can be configured using an FPGA, and the second data processing portion 514 can be configured using a GPU.

The first data processing portion 513 performs data processing with respect to the input imaging data, which is the imaging data from among the first existing imaging data (RAW data) to the third existing imaging data (RAW data) and the latest imaging data (RAW data), according to the type of the endoscope 2. As illustrated in FIG. 3, the first data processing portion 513 has a substantially same configuration as the configuration of the data processing portion 512 according to the embodiment described above. In the following explanation, for explanatory convenience, the first for-existing data processing portion 5121 according to the first modification example is referred to as a first for-existing data processing portion 5121A, the second for-existing data processing portion 5122 according to the first modification example is referred to as a second for-existing data processing portion 5122A, the third for-existing data processing portion 5123 according to the first modification example is referred to as a third for-existing data processing portion 5123A, and the common data processing portion 5124 according to the first modification example is referred to as a common data processing portion 5124A.

The first for-existing data processing portion 5121A performs data processing with respect to the first existing imaging data (RAW data), which is input thereto, according to a dedicated algorithm corresponding to the first existing endoscope 201. That algorithm corresponds to the first algorithm.

The data processing performed by the first for-existing data processing portion 5121A includes resolution adjustment, contrast adjustment, noise reduction, and brightness correction (gain correction) corresponding to the first existing endoscope 201.

The second for-existing data processing portion 5122A performs data processing with respect to the second existing imaging data (RAW data), which is input thereto, according to a dedicated algorithm corresponding to the second existing endoscope 202. That algorithm corresponds to the first algorithm.

The data processing performed by the second for-existing data processing portion 5122A includes resolution adjustment, contrast adjustment, noise reduction, and brightness correction (gain correction) corresponding to the second existing endoscope 202.

The third for-existing data processing portion 5123A performs data processing with respect to the third existing imaging data (RAW data), which is input thereto, according to a dedicated algorithm corresponding to the third existing endoscope 203. That algorithm corresponds to the first algorithm.

The data processing performed by the third for-existing data processing portion 5123A includes resolution adjustment, contrast adjustment, noise reduction, and brightness correction (gain correction) corresponding to the third existing endoscope 203.

The common data processing portion 5124A performs data processing with respect to the input imaging data, from among the first existing imaging data to the third existing imaging data (RAW data) and the latest imaging data (RAW data), according to an algorithm that is of a latter design generation than the first algorithm mentioned above and that is compatible to all endoscopes 2 including the first existing endoscope 201 to the third existing endoscope 203 and the latest endoscope 204. Herein, the algorithm represents the latest algorithm designed around the same time when the control device 5 was released or when the software representing the essential part of the control device 5 was updated; and corresponds to the second algorithm.

The data processing performed by the common data processing portion 5124A includes resolution adjustment, contrast adjustment, noise reduction, and brightness correction (gain correction) corresponding mainly to the latest endoscope 204. As explained above, the data processing is performed according to the algorithm that is compatible to all endoscopes 2 including the first existing endoscope 201 to the third existing endoscope 203 and the latest endoscope 204. For that reason, resolution adjustment, contrast adjustment, noise reduction, and brightness correction (gain correction) is applicable also to the first existing imaging data, the second existing imaging data, the third existing imaging data.

With respect to the imaging data that has been subjected to data processing by the first data processing portion 513, the second data processing portion 514 performs data processing that is not in accordance with the type of the endoscope 2, and generates a video signal based on that imaging data.

The data processing performed by the second data processing portion 514 includes image processing according to the display characteristics of the display 4 and includes, for example, color adjustment according to a user operation of the input unit 54. Examples of the display characteristics of the display 4 include the color balance and the gamma curve of the display 4.

Meanwhile, the data processing performed by the first data processing portion 513 or the data processing performed by the second data processing portion 514 can also include optical black reduction (clamping), white balance adjustment, color reconstruction, color correction matrix processing, gamma correction, and YC processing for converting RGB signals into luminance and chrominance signals (Y, Cb/Cr signals).

In FIG. 3, for explanatory convenience, the constituent elements that perform data processing according the algorithm that was used in the past (according to the first algorithm) (i.e., the first for-existing data processing portion 5121A to the third for-existing data processing portion 5123A) are illustrated using dots; and the constituent element that performs data processing according to the algorithm applicable to all endoscopes 2 including the first existing endoscope 201 to the third existing endoscope 203 and the latest endoscope 204 (according to the second algorithm) (i.e., the common data processing portion 5124A) is illustrated using oblique lines.

The medical image processing method according to the first modification example is identical to the medical image processing method according to the embodiment described above. Hence, that explanation is not given again.

Even when the configuration according to the first modification example is implemented, it is possible to achieve identical effects to the effects achieved according to the embodiment described above.

### Second Modification Example

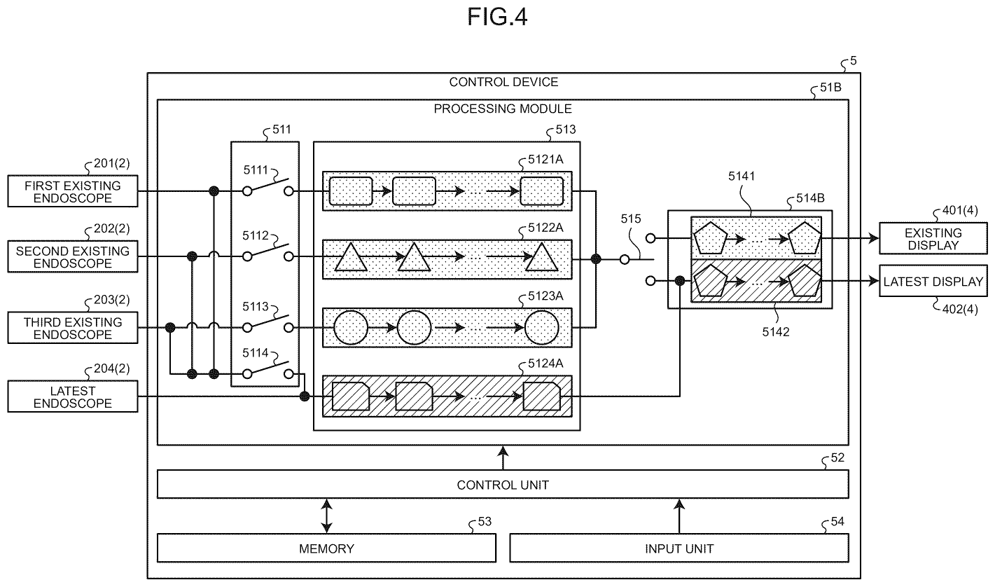

FIG. 4 is a diagram for explaining the second modification example of the embodiment. More particularly, FIG. 4 is a block diagram corresponding to FIG. 2.

Herein, a variety of displays 4 of different product generations are configured to be connectible to the control device 5 according to the second modification example. In the following explanation, from among the variety of displays 4, the existing of display 4 is referred to as an existing display 401 (see FIG. 4). Moreover, from among the variety of displays 4, the latest type of display 4 is referred to as a latest display 402 (see FIG. 4).

The latest display 402 either is a display of the same product generation as the control device 5 or is a display released around the same time when the software representing the essential part of the control device 5 was updated. The existing display 401 is a display of an earlier product generation than the product generation of the latest display 402.

The existing display 401 and the latest display 402 have mutually different display characteristics. Examples of the display characteristics include the color balance and the gamma curve.

In the embodiment described earlier, the configuration of the processing module 51 can be changed to the configuration according to the second modification example as illustrated in FIG. 4. In the following explanation, for explanatory convenience, the processing module 51 according to the second modification example is referred to as a processing module 51B.

As illustrated in FIG. 4, the processing module 51B includes a switching portion 515 and a second data processing portion 514B in addition to including the switching portion 511 and the first data processing portion 513 according to the first modification example described above.

Under the control performed by the control unit 52, the switching portion 515 switches the output destination for the first existing imaging data, which has been subjected to data processing by the first for-existing data processing portion 5121A, to the third existing imaging data, which has been subjected to data processing by the third for-existing data processing portion 5123A, between a for-existing data processing portion 5141 (see FIG. 4) and a common data processing portion 5142 (see FIG. 4) of the second data processing portion 514B.

In an identical manner to the second data processing portion 514 according to the first modification example described above, with respect to the imaging data that has been subjected to data processing by the first data processing portion 513, the second data processing portion 514B performs data processing that is not in accordance with the type of the endoscope 2, and generates a video signal based on that imaging data. As illustrated in FIG. 4, the second data processing portion 514B includes the for-existing data processing portion 5141 and the common data processing portion 5142.

With respect to the existing imaging data that is input from among the first existing imaging data, which has been subjected to data processing by the first for-existing data processing portion 5121A, to the third existing imaging data, which has been subjected to data processing by the third for-existing data processing portion 5123A, the for-existing data processing portion 5141 performs data processing that is not in accordance with the type of the endoscope 2; and generates a video signal based on the existing imaging data.

The data processing performed by the for-existing data processing portion 5141 includes image processing according to the display characteristics of the existing display 401 and includes, for example, color adjustment according to a user operation of the input unit 54.

With respect to the imaging data that is input from among the first existing imaging data, which has been subjected to data processing by the first for-existing data processing portion 5121A, to the third existing imaging data, which has been subjected to data processing by the third for-existing data processing portion 5123A, and the latest imaging data that has been processed by the common data processing portion 5124A, the common data processing portion 5142 performs data processing that is not in accordance with the type of the endoscope 2; and generates a video signal based on the existing imaging data.

The data processing performed by the common data processing portion 5142 includes image processing according to the display characteristics of the latest display 402 and includes, for example, color adjustment according to a user operation of the input unit 54.

In FIG. 4, for explanatory convenience, the constituent elements that perform data processing according the algorithm that was used in the past (according to the first algorithm) (i.e., the first for-existing data processing portion 5121A to the third for-existing data processing portion 5123A and the for-existing data processing portion 5141) are illustrated using dots; and the constituent element that performs data processing according to the algorithm applicable to all endoscopes 2 including the first existing endoscope 201 to the third existing endoscope 203 and the latest endoscope 204 (including the second algorithm) (i.e., the common data processing portions 5124A and 5142) is illustrated using oblique lines.

Given below is the explanation about the operations performed by the processing module 51B.

The operations performed by the switching portion 511 are identical to the operations performed according to the embodiment described earlier. Hence, the following explanation is mainly given about the operations performed by the switching portion 515 and the second data processing portion 514B.

Firstly, the explanation is given about the operations performed when the user performs the first existing endoscope 201.

As a result of a user operation of the input unit 54, when the existing display 401 is selected from among the existing display 401 and the latest display 402, under the control performed by the control unit 52, the switching portion 515 switches the output destination for the first existing imaging data, which has been subjected to data processing by the first for-existing data processing portion 5121A, to the for-existing data processing portion 5141. Then, the for-existing data processing portion 5141 performs data processing with respect to the first existing imaging data and generates a video signal. Subsequently, a display image based on the video signal is displayed in the existing display 401.

On the other hand, as a result of a user operation of the input unit 54, when the latest display 402 is selected from among the existing display 401 and the latest display 402, under the control performed by the control unit 52, the switching portion 515 switches the output destination for the first existing imaging data, which has been subjected to data processing by the first for-existing data processing portion 5121A, to the common data processing portion 5142. Then, the common data processing portion 5142 performs data processing with respect to the first existing imaging data and generates a video signal. Subsequently, a display image based on the video signal is displayed in the latest display 402.

Regarding the operations performed when the user uses the second existing endoscope 202, with reference to the explanation given above about “the operations performed when the user uses the first existing endoscope 201”; “the first for-existing data processing portion 5121A” can be substituted with “the second for-existing data processing portion 5122A”, and “the first existing imaging data” can be substituted with “the second existing imaging data”.

Moreover, regarding the operations performed when the user uses the third existing endoscope 203, with reference to the explanation given above about “the operations performed when the user uses the first existing endoscope 201”; “the first for-existing data processing portion 5121A” can be substituted with “the third for-existing data processing portion 5123A”, and “the first existing imaging data” can be substituted with “the third existing imaging data”.

Given below is the explanation about the operations performed when the user uses the latest endoscope 204.

The latest imaging data that has been subjected to data processing by the common data processing portion 5124A is directly input to the common data processing portion 5142 without involving the switching portion 515. Then, the common data processing portion 5142 performs data processing with respect to the latest imaging data and generates a video signal. Subsequently, a display image based on the video signal is displayed in the latest display 402.

According to the second modification example explained above, in addition to achieving identical effects to the effects achieved according to the embodiment described earlier, it becomes possible to achieve the following effects.

According to the second modification example, it becomes possible to generate appropriate endoscope images compatible to various type of displays 4, thereby enabling achieving further enhancement in the user-friendliness.

### Third Modification Example

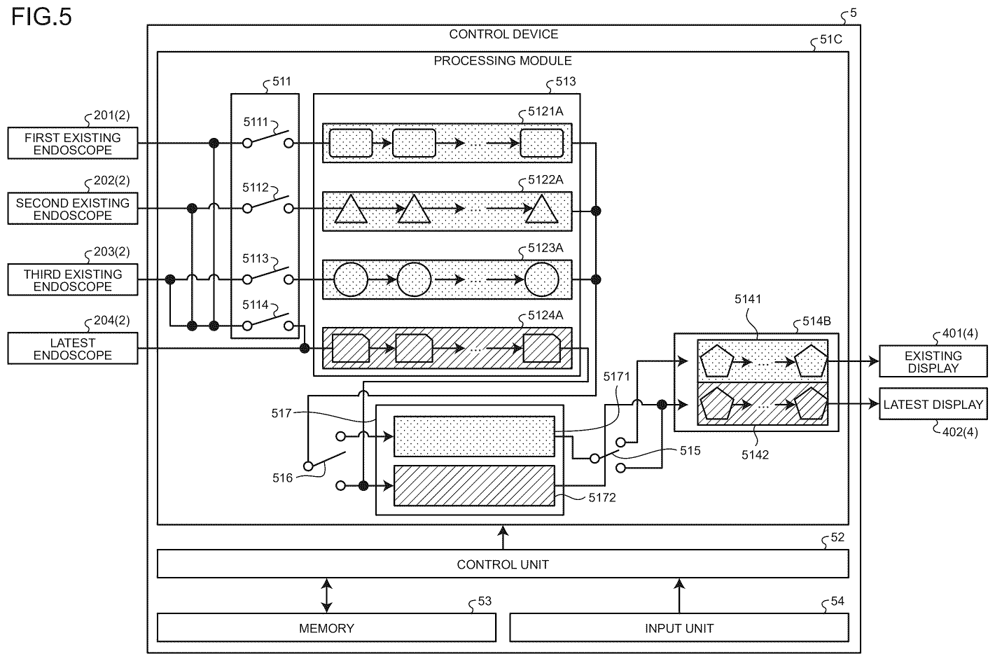

FIG. 5 is a diagram for explaining a third modification example of the embodiment. More particularly, FIG. 5 is a block diagram corresponding to FIG. 2.

In the second modification example explained above, the configuration of the processing module 51B can be changed to a configuration according to the third modification example as illustrated in FIG. 5. In the following explanation, for explanatory convenience, the processing module 51B according to the third modification example is referred to as a processing module 51C.

As illustrated in FIG. 5, the processing module 51C includes a switching portion 516 and a third data processing portion 517 in addition to including the switching portions 511 and 515, the first data processing portion 513, and the second data processing portion 514B according to the second modification example.

Under the control performed by the control unit 52, the switching portion 16 switches the output destination for the imaging data from the first existing imaging data to the third existing imaging data, which has been subjected to data processing by the first for-existing data processing portion 5121A to the third for-existing data processing portion 5123A, between a for-existing data processing portion 5171 (see FIG. 5) and a common data processing portion 5172 (see FIG. 5) of the third data processing portion 517.

With respect to the imaging data that has been subjected to data processing by the first data processing portion 513, the third data processing portion 517 performs data processing corresponding to the setting mode that is set from among a plurality of setting modes meant for providing the diagnosis support. The setting modes include at least one of a normal light observation mode in which a white light is irradiated inside the subject; a special light observation mode in which either an excitation light that is a narrow-band light meant for causing excitation of a fluorescent agent, such as indocyanine green, inside the subject or a narrow-band light used in narrow band imaging (NBI) is irradiated; or a structural emphasis observation mode in which structural emphasis is carried out by performing image processing. The setting mode from among the setting modes is set, for example, by a user operation of the input unit 54. As illustrated in FIG. 5, the third data processing portion 517 includes a for-existing data processing portion 5171 and a common data processing portion 5172.

With respect to the existing imaging data that is input from among the first existing imaging data to the third existing imaging data which has been subjected to data processing by the first for-existing data processing portion 5121A to the third for-existing data processing portion 5123A, the for-existing data processing portion 5171 performs data processing corresponding to the setting mode that is set.

Herein, the data processing performed by the for-existing data processing portion 5171 corresponds to the setting mode that was used in a control device of an earlier product generation (hereinafter, referred to as a conventional control device) than the product generation of the control device 5. For example, in a conventional control device, when only the normal light observation mode is provided from among a variety of observation nodes, the data processing performed by the for-existing data processing portion 5171 represents image processing corresponding to the normal light observation mode. That is, the data processing performed by the for-existing data processing portion 5171 is performed according to an existing algorithm.

As illustrated in FIG. 5, under the control performed by the control unit 52, the switching portion 515 according to the third modification example switches the output destination for the imaging data from the first existing imaging data to the third existing imaging data, which has been subjected to data process by the for-existing data processing portion 5171, between the for-existing data processing portion 5141 and the common data processing portion 5142 of the second data processing portion 514B.

With respect to the imaging data that is input from among the first existing imaging data to the third existing imaging data and the latest imaging data which has been subjected to data processing by the first for-existing data processing portion 5121A to the third for-existing data processing portion 5123A and the common data processing portion 5124A, the common data processing portion 5172 performs data processing corresponding to the setting mode that is set.

Herein, the data processing performed by the common data processing portion 5172 corresponds to the setting mode that was used in a control device of an earlier product generation (hereinafter, referred to as a conventional control device) than the product generation of the control device 5, as well as corresponds to the setting mode that is newly set at the time of release of the control device 5. For example, when the newly-set setting mode is a special light observation mode in which an excitation light that is a narrow-band light of a new wavelength bandwidth is used for causing excitation of a new fluorescent agent, the data processing performed by the common data processing portion 5172 includes image processing corresponding to that special light observation mode. When the newly-set setting mode is a structural emphasis observation mode, the data processing performed by the common data processing portion 5172 includes filter processing for structure emphasis. Thus, the data processing performed by the common data processing portion 5172 is performed according to the latest algorithm.

In FIG. 2, for explanatory convenience, the constituent elements that perform data processing according the algorithm that was used in the past (including the first algorithm) (i.e., the first for-existing data processing portion 5121A to the third for-existing data processing portion 5123A and the for-existing data processing portions 5141 and 5171) are illustrated using dots; and the constituent elements that perform data processing according to the algorithm applicable to all endoscopes 2 including the first existing endoscope 201 to the third existing endoscope 203 and the latest endoscope 204 (including the second algorithm) (i.e., the common data processing portions 5124A, 5142, and 5172) are illustrated using oblique lines.

Given below is the explanation about the operations performed by the processing module 51C.

The operations performed by the switching portions 511 and 515 are identical to the operations performed according to the embodiment and the second modification example described above. Hence, the following explanation is mainly given about the operations performed by the switching portion 516 and the third data processing portion 517.

Firstly, the explanation is given about the operations performed when the user uses the first existing endoscope 201.

As a result of a user operation of the input unit 54, when the existing algorithm is selected from among the existing algorithm and the latest algorithm, under the control performed by the control unit 52, the switching portion 516 switches the output destination for the first existing imaging data, which has been subjected to data processing by the first for-existing data processing portion 5121A, to the for-existing data processing portion 5171. Then, the for-existing data processing portion 5171 performs data processing with respect to the first existing imaging data according to the existing algorithm corresponding to the setting mode that is set.

On the other hand, as a result of a user operation of the input unit 54, when the latest algorithm is selected from among the existing algorithm and the latest algorithm, under the control performed by the control unit 52, the switching portion 516 switches the output destination for the first existing imaging data, which has been subjected to data processing by the first for-existing data processing portion 5121A, to the common data processing portion 5172. Then, the common data processing portion 5172 performs data processing with respect to the first existing imaging data according to the latest algorithm corresponding to the setting mode that is set.

Regarding the operations performed when the user uses the second existing endoscope 202, with reference to the explanation given above about “the operations performed when the user uses the first existing endoscope 201”; “the first for-existing data processing portion 5121A” can be substituted with “the second for-existing data processing portion 5122A”, and “the first existing imaging data” can be substituted with “the second existing imaging data”.

Regarding the operations performed when the user uses the third existing endoscope 203, with reference to the explanation given above about “the operations performed when the user uses the first existing endoscope 201”; “the first for-existing data processing portion 5121A” can be substituted with “the third for-existing data processing portion 5123A”, and “the first existing imaging data” can be substituted with “the third existing imaging data”.

Given below is the explanation about the operations performed when the user uses the latest endoscope 204.

The latest imaging data that has been subjected to data processing by the common data processing portion 5124A is directly input to the common data processing portion 5172 without involving the switching portion 516. Then, the common data processing portion 5172 performs data processing with respect to the latest imaging data according to the latest algorithm corresponding to the setting mode.

According to the third modification example described above, in addition to achieving identical effects to the effects achieved according to the embodiment and the second modification example described earlier, it becomes possible to achieve the following effects.

According to the third modification example, it becomes possible to generate appropriate endoscope images corresponding to new observation modes meant for providing the diagnosis support, thereby enabling achieving further enhancement in the user-friendliness.

Meanwhile, the third data processing portion 517 can perform data processing at an earlier stage than the data processing performed by the first data processing portion 513.

### Fourth Modification Example

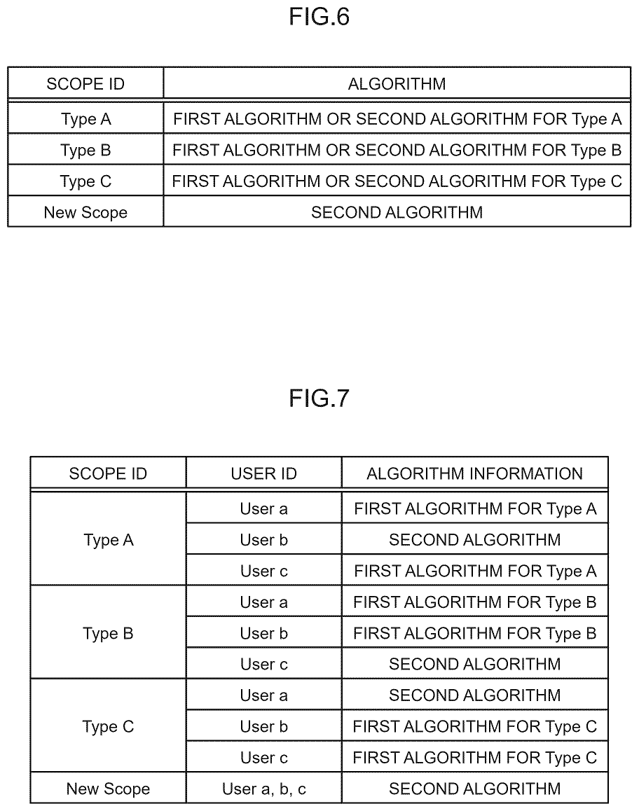

FIG. 6 is a diagram for explaining the fourth modification example according to the embodiment.

In the embodiment described earlier, at the time of making the user select between the first algorithm and the second algorithm, the algorithm to be selected can be displayed in the display 4 based on a scope ID (identifier).

More particularly, the scope ID represents unique identification information of the endoscope 2, which is connected to the control device 5, for enabling identification of that endoscope 2; and corresponds to device identification information. As illustrated in FIG. 6, “Type A” represents the scope ID of the first existing endoscope 201. That scope ID is stored in the memory (not illustrated) which is installed in the first existing endoscope 201. Similarly, “Type B” represents the scope ID of the second existing endoscope 202. That scope ID is stored in the memory (not illustrated) which is installed in the second existing endoscope 202. Moreover, “Type C” represents the scope ID of the third existing endoscope 203. That scope ID is stored in the memory (not illustrated) which is installed in the third existing endoscope 203. Furthermore, “New Scope” represents the scope ID of the latest endoscope 204. That scope ID is stored in the memory (not illustrated) which is installed in the latest endoscope 204.

The control unit 52 according to the fourth modification example reads the scope ID from the memory (not illustrated) of the endoscope 2 that is connected to the control device 5, and determines the type of that endoscope 2 (determination step). When the type of the endoscope 2 is determined to be the first existing endoscope 201 (i.e., when the scope ID is “Type A”), as illustrated in FIG. 6, the control unit 52 displays “first algorithm or second algorithm for Type A” in the display 4 for enabling selection of the algorithm. Similarly, when the type of the endoscope 2 is determined to be the second existing endoscope 202 (i.e., when the scope ID is “Type B”), the control unit 52 displays “first algorithm or second algorithm for Type B” in the display 4 for enabling selection of the algorithm. Moreover, when the type of the endoscope 2 is determined to be the third existing endoscope 203 (i.e., when the scope ID is “Type C”), the control unit 52 displays “first algorithm or second algorithm for Type C” in the display 4 for enabling selection of the algorithm. Furthermore, when the type of the endoscope 2 is determined to be the latest endoscope 204 (i.e., when the scope ID is “New Scope”), the control unit 52 displays “second algorithm” in the display 4 for indicating the algorithm to be implemented. Thus, the control unit 52 according to the fourth modification example functions as a determining portion.

According to the fourth modification example described above, in addition to achieving identical effects to the effects achieved according to the embodiment described earlier, it becomes possible to achieve the following effects.

According to the fourth modification example, the selectable algorithms can be presented to the user, thereby enabling achieving further enhancement in the user-friendliness.

### Fifth Modification Example

FIG. 7 is a diagram for explaining the fifth modification example of the embodiment.

In the embodiment described earlier, at the time of controlling the operations of the switching portion 511, the control unit 52 can control those operations based on reference information stored in the memory 53.

More particularly, in the memory 53, the control unit 52 stores reference information in which the following information obtained during previous usage of the endoscope system 1 are associated with each other: the scope IDs; the user IDs of the users who used the endoscopes 2; and algorithm information indicating the implemented algorithms from among the first algorithm and the second algorithm. Herein, the scope IDs represent the scope IDs explained earlier in the fourth modification example. The user IDs represent user-specific identification information enabling identification of the users who used the endoscopes 2. For example, a user ID is input as a result of a user operation of the input unit 54, and corresponds to user identification information. The reference information stored in the memory 53 is illustrated in FIG. 7.

Then, from the reference information stored in the memory 53, the control unit 52 recognizes the algorithm information that is associated to both of the scope ID and the user ID that are obtained during the current usage of the endoscope system 1 (recognition step). Moreover, the control unit 52 controls the operations of the switching portion 511 to ensure that the processing module 51 performs data processing according to the algorithm corresponding to the recognized algorithm information. For example, when “type A” and “User a” are obtained as the scope ID and the user ID, respectively, during the current usage of the endoscope system 1; the control unit 52 recognizes, from the reference information, “first algorithm for Type A” as the algorithm information associated to the scope ID and the user ID that are obtained. Then, in order to ensure that the processing module 51 performs data processing according to “first algorithm for Type A”, the control unit 52 sets the first for-existing switching portion 5111 to the “ON” state and sets the common switching portion 5114 to the “OFF” state. Thus, the control unit 52 according to the fifth modification example functions as a recognizing portion.

According to the fifth modification example described above, in addition to achieving identical effects to the effects achieved according to the embodiment described earlier, it becomes possible to achieve the following effects.

According to the fifth embodiment, if either the first algorithm or the second algorithm has been selected once in the past, it gets automatically selected there onwards. As a result, it becomes possible to eliminate the time and efforts required for making the user select an algorithm, thereby enabling achieving further enhancement in the user-friendliness.

Meanwhile, if the configuration according to the fifth modification example is to be implemented in the configuration according to the second modification example, the reference information in which display information indicating the display selected from among the existing display 401 and the latest display 402 in addition to the scope IDs obtained during the previous usage of the endoscope system 1, the user IDs enabling identification of the users who used the endoscopes 2 during the previous usage of the endoscope system 1, and the algorithm information indicating the implemented algorithms from among the first algorithm and the second algorithm during the previous usage of the endoscope system 1 are associated with each other can be also used.

Thus, from the reference information stored in the memory 53, the control unit 52 recognizes the algorithm information and the display information associated to both of the scope ID and the user ID that are obtained during the current usage of the endoscope system 1. Then, the control unit 52 controls the operations of the switching portion 511 to ensure that the processing module 51 performs data processing according to the algorithm corresponding to the recognized algorithm information. Moreover, the control unit 52 controls the operations of the switching portion 515 to ensure that the display images are displayed in the display 4 corresponding to the recognized display information.

### Sixth Modification Example

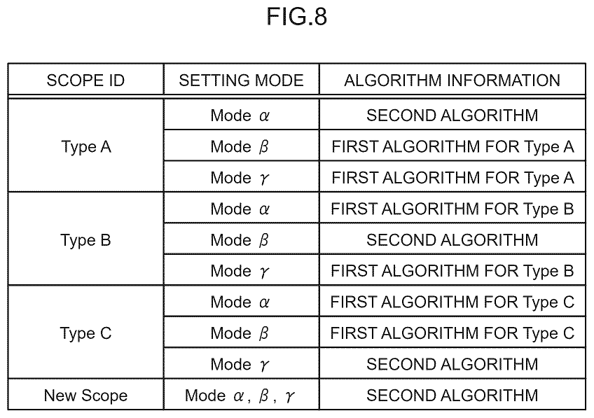

FIG. 8 is a diagram for explaining the sixth modification example of the embodiment.

In the third modification example described earlier, at the time of controlling the operations of the switching portion 511, the control unit 52 can control those operations based on reference information stored in the memory 53.

More particularly, in the memory 53, the control unit 52 stores reference information in which the following information obtained during the previous usage of the endoscope system 1 are associated with each other: the scope IDs; mode information indicating the setting mode that is set from among a plurality of setting modes meant for providing the diagnosis support; and algorithm information indicating the implemented algorithms from among the first algorithm and the second algorithm. Herein, the scope IDs represent the scope IDs explained earlier in the fourth modification example. The reference information stored in the memory 53 is illustrated in FIG. 8.

Then, from the reference information stored in the memory 53, the control unit 52 recognizes the algorithm information associated to both of the scope ID and the mode information that are obtained during the current usage of the endoscope system 1 (recognition step). Moreover, the control unit 52 controls the operations of the switching portion 511 to ensure that the processing module 51B performs data processing according to the algorithm corresponding to the recognized algorithm information. For example, when “type A” and “Mode a” are obtained as the scope ID and the mode information, respectively, during the current usage of the endoscope system 1; the control unit 52 recognizes, from the reference information, “second algorithm” as the algorithm information associated to the scope ID and the mode information. Then, in order to ensure that the processing module 51B performs data processing according to “second algorithm”, the control unit 52 sets the first for-existing switching portion 5111 to the “OFF” state and sets the common switching portion 5114 to the “ON” state. Thus, the control unit 52 according to the sixth modification example functions as a recognizing portion.

Even when the configuration according to the sixth modification is implemented, it is possible to achieve identical effects to the effects achieved according to the third and fifth modification examples.

### Seventh Modification Example

In the sixth modification example, the reference information in which second algorithm information indicating the algorithm selected from among the existing algorithms and the latest algorithm, and display information indicating the display selected from among the existing display 401 and the latest display 402 in addition to the scope IDs obtained during the previous usage of the endoscope system 1, the mode information indicating the setting mode that is set from among a plurality of setting modes meant for providing the diagnosis support during the previous usage of the endoscope system 1, and the algorithm information indicating the implemented algorithms from among the first algorithm and the second algorithm during the previous usage of the endoscope system 1 are associated with each other can be also used.

Thus, from the reference information stored in the memory 53, the control unit 52 recognizes the algorithm information, the second algorithm information, and the display information associated to both of the scope ID and the mode information that are obtained during the current usage of the endoscope system 1. Then, the control unit 52 controls the operations of the switching portion 511 to ensure that the processing module 51B performs data processing according to the algorithm corresponding to the recognized algorithm information. Moreover, the control unit 52 controls the operations of the switching portion 516 to ensure that the processing module 51B performs data processing according to the algorithm corresponding to the recognized second algorithm information. Furthermore, the control unit 52 controls the operations of the switching portion 515 to ensure that the display images are displayed in the display 4 corresponding to the recognized display information.

Even when the configuration according to the seventh modification example is implemented, it is possible to achieve identical effects to the effects achieved according to the sixth modification example described above.

### Eighth Modification Example

In the sixth modification example described earlier, the reference information in which the user IDs in addition to the scope IDs obtained during the previous usage of the endoscope system 1, the mode information indicating the setting mode that is set from among a plurality of setting modes meant for providing the diagnosis support during the previous usage of the endoscope system 1, and the algorithm information indicating the implemented algorithms from among the first algorithm and the second algorithm during the previous usage of the endoscope system 1 are associated with each other can be also be used.

Thus, from the reference information stored in the memory 53, the control unit 52 recognizes the algorithm information associated to the scope ID, the mode information, and the user ID that are obtained during the current usage of the endoscope system 1 (recognition step). Then, the control unit 52 controls the operations of the switching portion 511 to ensure that the processing module 51B performs data processing according to the algorithm corresponding to the recognized algorithm information.

Even when the configuration according to the eighth modification example is implemented, it is possible to achieve identical effects to the effects achieved according to the sixth modification example described above.

### Ninth Embodiment

In the embodiment and in the first to eighth modification examples described above, the number of for-existing data processing portions (the first for-existing data processing portion 5121 (5121A) to the third for-existing data processing portion 5123 (5123A)) is not limited to three, and it is possible to have a different number of for-existing data processing portions. The number of for-existing data processing portions can be decided in accordance with the number of existing endoscopes (the first existing endoscope 201 to the third existing endoscope 203). Moreover, in the second and third modification examples described above, it is not limited to have only a single for-existing data processing portion 5141 and a single for-existing data processing portion 5171, respectively. Thus, it is possible to have a different number of for-existing data processing portions.

### 10-Th Modification Example

In the embodiment and in the first to ninth modification examples described above, the medical image processing device is installed in the endoscope system 1 in which the endoscope 2 is configured using a flexible endoscope. However, that is not the only possible case. Alternatively, for example, the medical image processing device can be installed in an endoscope system in which the endoscope 2 is configured using a rigid endoscope. Moreover, the medical image processing device can be installed in a medical observation system that includes a surgical microscope that expands, for observation purposes, a predetermined field of view of the inside the photographic subject (inside the biological object) or on the surface of the photographic subject (on the surface of the biological object) (for example, refer to Japanese Patent Application Laid-open No. 2016-42981). In that case, the surgical microscope corresponds to the medical observation device.

The medical image processing device, the medical image processing method, and the computer program product according to the disclosure enable achieving enhancement in the user-friendliness.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A medical image processing device comprising a processing module performing data processing with respect to imaging data which is input, the processing module being capable of implementing a first algorithm which enables data processing with respect to first imaging data obtained as a result of imaging performed by a first endoscope, and a second algorithm which enables data processing with respect to the first imaging data and with respect to second imaging data obtained as a result of imaging performed by a second endoscope,
the processing module being configured to
determine type of endoscope that is connected,
when connection with the first endoscope is determined, perform data processing according to an algorithm that is selected as a result of a user operation from among the first algorithm and the second algorithm, and
when connection with the second endoscope is determined, perform data processing according to the second algorithm.

2. The medical image processing device according to claim 1, wherein
the first algorithm is an algorithm dedicated for the first endoscope, and
the second algorithm is an algorithm of a latter design generation than the first algorithm, and is compatible to the first endoscope and to the second endoscope which is an endoscope of a latter product generation than the first endoscope.

3. A medical image processing device comprising:
a processing module performing data processing with respect to imaging data obtained as a result of imaging performed by a medical observation device; and
an operation receiver receiving a user operation, processing module being configured to
with respect to first image data obtained as a result of imaging performed by a first medical observation device from among medical observation devices, perform data processing according to an algorithm that is selected in the user operation from among
a first algorithm dedicated for the first medical observation device, and
a second algorithm that is an algorithm of a latter design generation than the first algorithm and that is compatible to the first medical observation device and to a second medical observation device which is the medical observation device of a latter product generation than the first medical observation device, and
with respect to second image data obtained as a result of imaging performed by the second medical observation device, perform data processing according to the second algorithm.

4. The medical image processing device according to claim 3, wherein the processing module includes
a first data processing portion configured to perform data processing according to type of the medical observation device with respect to either the first imaging data or the second imaging data, and
a second data processing portion configured to perform data processing that is not in accordance with type of the medical observation device, with respect to either the first imaging data having been subjected to data processing by the first data processing portion or the second imaging data having been subjected to data processing by the first data processing portion.

5. The medical image processing device according to claim 4, wherein the data processing according to type of the medical observation device includes at least one of noise reduction or brightness correction according to type of the medical observation device.

6. The medical image processing device according to claim 4, wherein the data processing that is not in accordance with type of the medical observation device includes image processing according to display characteristics of a display that is connected to the medical image processing device.

7. The medical image processing device according to claim 4, wherein the processing module further includes a third data processing portion configured to perform data processing according to a setting mode that is set from among a plurality of setting modes meant for providing diagnosis support, with respect to either the first imaging data or the second imaging data.

8. The medical image processing device according to claim 3, further comprising a determining portion configured to determine type of the medical observation device connected to the medical image processing device, based on device identification information which is unique to the medical observation device connected to the medical image processing device and which enables identification of the medical observation device, wherein
when the determining portion determines that the first medical observation device represents the medical observation device connected to the medical image processing device, the processing module is configured to perform data processing according to an algorithm selected in the user operation from among the first algorithm and the second algorithm, with respect to the first imaging data, and
when the determining portion determines that the second medical observation device represents the medical observation device connected to the medical image processing device, the processing module is configured to perform data processing according to the second algorithm with respect to the second imaging data.

9. The medical image processing device according to claim 3, further comprising:
a memory that stores therein reference information in which
device identification information that enables identification of the medical observation device connected to the medical image processing device during previous usage of the medical image processing device,
user identification information that enables identification of a user who used the medical observation device during the previous usage of the medical image processing device, and
algorithm information that indicates an algorithm which was used from among the first algorithm and the second algorithm during the previous usage of the medical image processing device
are associated with each other; and
a recognizing portion configured to recognize, from the reference information, the algorithm information associated to both of the device identification information obtained during current usage of the medical image processing device and the user identification information obtained during the current usage of the medical image processing device, wherein
during the current usage of the medical image processing device, with respect to either the first imaging data or the second imaging data, the processing module is configured to perform data processing according to an algorithm that is specified in the algorithm information recognized by the recognizing portion.

10. The medical image processing device according to claim 3, further comprising:
a memory configured to store therein reference information in which
device identification information that enables identification of the medical observation device connected to the medical image processing device during previous usage of the medical image processing device,
mode information that indicates a setting mode which is set from among a plurality of setting modes meant for providing diagnosis support during the previous usage of the medical image processing device, and
algorithm information that indicates an algorithm which was used from among the first algorithm and the second algorithm during the previous usage of the medical image processing device
are associated with each other; and
a recognizing portion configured to recognize, from the reference information, the algorithm information associated to both of the device identification information obtained during current usage of the medical image processing device and the mode information obtained during the current usage of the medical image processing device, wherein
with respect to either the first imaging data or the second imaging data, the processing module is configured to perform data processing according to an algorithm that is specified in the algorithm information recognized by the recognizing portion.

11. The medical image processing device according to claim 3, further comprising:
a memory configured to store therein reference information in which
device identification information that enables identification of the medical observation device connected to the medical image processing device during previous usage of the medical image processing device,
user identification information that enables identification of a user who used the medical observation device during the previous usage of the medical image processing device,
mode information that indicates a setting mode which is set from among a plurality of setting modes meant for providing diagnosis support during the previous usage of the medical image processing device, and
algorithm information that indicates an algorithm which was used from among the first algorithm and the second algorithm during the previous usage of the medical image processing device
are associated with each other; and
a recognizing portion configured to recognize, from the reference information, the algorithm information associated to the device identification information, the user identification information, and the mode information that are obtained during current usage of the medical image processing device, wherein
with respect to either the first imaging data or the second imaging data, the processing module is configured to perform data processing according to an algorithm that is specified in the algorithm information recognized by the recognizing portion.

12. The medical image processing device according to claim 10, wherein the plurality of setting modes include at least one of
a normal light observation mode in which a normal light is irradiated onto a subject,
a special light observation mode in which a narrow-band light is irradiated onto the subject, or
a structural emphasis observation mode in which image processing is performed to perform structural emphasis.

13. The medical image processing device according to claim 4, wherein the first data processing portion as well as the second data processing portion include either a field-programmable gate array (FPGA) or a graphics processing unit (GPU).

14. The medical image processing device according to claim 13, wherein
the first data processing portion includes the FPGA, and
the second data processing portion includes the GPU.

15. The medical image processing device according to claim 3, wherein the medical image observation device is an endoscope configured to be inserted inside of a subject and perform imaging of the inside of the subject to generate the imaging data.

16. A medical image processing method implemented in a medical image processing device, the method comprising:
with respect to first imaging data which is obtained as a result of imaging performed by a first medical observation device, performing data processing according to an algorithm selected in a user operation from among
a first algorithm dedicated for the first medical observation device, and
a second algorithm that is an algorithm of a latter design generation than the first algorithm and that is compatible to the first medical observation device and to a second medical observation device which is a latter product generation than the first medical observation device; and
with respect to second imaging data which is obtained as a result of imaging performed by the second medical observation device, performing data processing according to the second algorithm.

17. The medical image processing method according to claim 16, further comprising:
when it is determined that, based on device identification information which is unique to a medical observation device connected to the medical image processing device and which enables identification of the medical observation device, the first medical observation device represents the medical observation device connected to the medical image processing device, performing data processing according to an algorithm selected in the user operation from among the first algorithm and the second algorithm, with respect to the first imaging data; and
when it is determined that, based on the device identification information, the second medical observation device represents the medical observation device connected to the medical image processing device, performing data processing data according to the second algorithm with respect to the second imaging.

18. The medical image processing method according to claim 16, further comprising:
recognizing, from reference information in which
device identification information that enables identification of the medical observation device connected to the medical image processing device during previous usage of the medical image processing device,
user identification information that enables identification of a user who used the medical observation device during the previous usage of the medical image processing device, and
algorithm information that indicates an algorithm which was used from among the first algorithm and the second algorithm during the previous usage of the medical image processing device
are associated with each other,
the algorithm information associated to both of the device identification information obtained during current usage of the medical image processing device and the user identification information obtained during the current usage of the medical image processing device; and
during the current usage of the medical image processing device, with respect to either the first imaging data or the second imaging data, performing data processing according to an algorithm that is specified in the recognized algorithm.

19. The medical image processing method according to claim 16, further comprising:
recognizing, from reference information in which
device identification information that enables identification of the medical observation device connected to the medical image processing device during previous usage of the medical image processing device,
mode information that indicates a setting mode which is set from among a plurality of setting modes meant for providing diagnosis support during the previous usage of the medical image processing device, and
algorithm information that indicates an algorithm which was used from among the first algorithm and the second algorithm during the previous usage of the medical image processing device
are associated with each other,
the algorithm information associated to both of the device identification information obtained during current usage of the medical image processing device and the mode information obtained during the current usage of the medical image processing device; and
with respect to either the first imaging data or the second imaging data, performing data processing according to an algorithm that is specified in the recognized algorithm information.

20. A non-transitory computer-readable recording medium with an executable program stored thereon, the program causing a medical image processing device to execute:
with respect to first imaging data which is obtained as a result of imaging performed by a first medical observation device, performing data processing according to an algorithm selected in a user operation from among
a first algorithm dedicated for the first medical observation device, and
a second algorithm that is an algorithm of a latter design generation than the first algorithm and that is compatible to the first medical observation device and to a second medical observation device which is a latter product generation than the first medical observation device; and
with respect to second imaging data which is obtained as a result of imaging performed by the second medical observation device, performing data processing according to the second algorithm.

21. The recording medium according to claim 20, wherein the program causes the medical image processing device to further execute:
when it is determined that, based on device identification information which is unique to a medical observation device connected to the medical image processing device and which enables identification of the medical observation device, the first medical observation device represents the medical observation device connected to the medical image processing device, performing data processing according to an algorithm selected in the user operation from among the first algorithm and the second algorithm, with respect to the first imaging data; and
when it is determined that, based on the device identification information, the second medical observation device represents the medical observation device connected to the medical image processing device, performing data processing data according to the second algorithm with respect to the second imaging.

22. The recording medium according to claim 20, wherein the program causes the medical image processing device to further execute:
recognizing, from reference information in which
device identification information that enables identification of the medical observation device connected to the medical image processing device during previous usage of the medical image processing device,
user identification information that enables identification of a user who used the medical observation device during the previous usage of the medical image processing device, and
algorithm information that indicates an algorithm which was used from among the first algorithm and the second algorithm during the previous usage of the medical image processing device
are associated with each other,
the algorithm information associated to both of the device identification information obtained during current usage of the medical image processing device and the user identification information obtained during the current usage of the medical image processing device; and
during the current usage of the medical image processing device, with respect to either the first imaging data or the second imaging data, performing data processing according to an algorithm that is specified in the recognized algorithm.
